(19) United States
(12) Patent Application Publication
(10) Pub. No.: US 2020/0229846 A1
(43) Pub. Date: Jul. 23, 2020

(54) Device for applying compression force on the uterine cervix

(71) Applicant: Humberto Jose Azpurua-Pardi, Caracas (VE)

(72) Inventor: Humberto Jose Azpurua-Pardi, Caracas (VE)

(21) Appl. No.: 16/503,503

(22) Filed: Jul. 4, 2019

Related U.S. Application Data
(60) Provisional application No. 62/795,592, filed on Jan. 23, 2019.

(51) Int. Cl. A61B 17/42; A61B 17/12

(52) U.S. Cl. CPC A61B 17/42; A61B 17/12013; A61B 17/1322; A61B 2017/4225; A61B 2017/12004

(57) ABSTRACT

Device for applying compression force around the uterine cervix in different conditions in obstetrics and gynaecology, for example, as a tourniquet to stop a haemorrhage, consisting of a long hollow component (102) that has a proximal end (106) and a distal end (108), and a flexible component (110) that has a length greater than the length of the hollow component (102) and that is inserted by its ends (112, 114) in the hollow of the hollow component (102) forming a loop (120) of a variable diameter that emerges through the distal end (108), apt for wrapping around the uterine cervix and applying compression on it when at least one of the ends (114) of the flexible component (110) is pulled from the proximal end (106) of the hollow component (102).

### CROSS-REFERENCE TO RELATED APPLICATIONS

Not Applicable

### STATEMENT REGARDING FEDERALLY SPONSORED RESEARCH OR DEVELOPMENT

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT INCORPORATION-BY-REFERENCE OF MATERIAL SUBMITTED ON A COMPACT DISC

Not Applicable

### BACKGROUND OF THE INVENTION TECHNICAL FIELD

### Field of the Invention

The field of application of this invention is framed within the sector of the industry dedicated to the manufacture of medical equipment and devices. More specifically, the device described herein refers to medical devices that apply compression on the uterine cervix during medical procedures.

### Description of Related Art

During surgery, bleeding in the surgical site is a problem. Bleeding is generally controlled by electro-cauterisation, applying haemostatic forceps, suturing them with sutures or applying low pressure gauze and maintaining the pressure during some time until a clot is formed and the bleeding is controlled. A tourniquet is a constriction or compression device used to control the arterial and venous blood flow in a limb or organ during a short period of time. Temporary tourniquets have been used for a long time in medicine and are truly useful in controlling bleeding, but their location depends on the anatomical structure to be dealt with. Due to its anatomical position, up to now no mechanism/device has been described for the placement of a tourniquet on the uterine cervix.

Thus, there exists a need that has not yet been solved for a mechanism/device that controls bleeding during surgery on the cervix. More specifically, there is a need felt but not solved for a device that applies compression to the cervix to stop the haemorrhage in a substantially instantaneous manner, with the development of a device for this purpose being the objective of this invention.

Furthermore, and in reference to the current state of the art, it should be noted that, by document CN205072950(U), there is a device that applies compression to the uterine cervix which, however, has a structural configuration and technical characteristics markedly different from the device of the present invention. In particular, this document refers to a cervical annular tourniquet provided with a bridge type strip. One end of an elastic rope is connected respectively at both ends forming a ring. The utility model disclosed can tighten the cervix reducing effectively the bleeding in the conization of cervix, and consequently the need of haemostatic sutures avoiding secondary complications to the patient.

Moreover this application claims priority to and the benefit of the provisional/non-provisional patent application titled “Methods and Apparatus for Providing a Compressive Force to The Uterine Cervix”, application No. 62/795,592, filed in the United States Patent and trademark Office on Jan. 23, 2019. The specification of the above referenced patent application is incorporated herein by reference in its entirety.

### BRIEF SUMMARY OF THE INVENTION

The invention, as expressed in the title of this descriptive report, refers to a device for applying compression force on the uterine cervix that provides advantages and characteristics to the function for which it is intended, which are described in detail later, and which signify an improvement on the state of the art.

More specifically, the purpose of the invention is focused on a device, which is essentially formed by a long hollow component and a flexible component, in the form of a rope, strap or cord, that is inserted in the hollow component with, at least, one end emerging from the proximal end of the hollow component and forming a loop on the opposite distal end, and serves to apply compression force around the uterine cervix, for different conditions in obstetrics and gynaecology, for instance as a tourniquet to stop a haemorrhage.

### BRIEF DESCRIPTION OF THE DRAWINGS

In order to complement the description that is being made and for the purpose of aiding a better understanding of the characteristics of the invention, a set of plans are attached to this descriptive report, as an integral part thereof, in which the following are represented as an illustration. These embodiments and other aspects of the invention will be readily apparent from the description and the appended drawings, which are meant to illustrate and not to limit the invention. The drawings are not to scale and emphasis is generally being placed on illustrating the principles of the invention.

FIGS. 1A, 1B, 1C, 1D, 1E and 1F: show views of a first embodiment of the device of the invention where more specifically:

FIG. 1A is a perspective of the long hollow component,

FIG. 1B is a longitudinal section of the long hollow component with a flexible component or rope in the insertion phase through its first end,

FIG. 1C is a section similar to the previous one with an indication of the insertion of the second end of the flexible component (rope),

FIG. 1D is a perspective of the long component with the flexible component (rope) completely incorporated, forming the loop,

and FIGS. 1E and 1F are views in longitudinal section, partial and complete, respectively, of the flexible component or rope once it is inserted completely in the long hollow component showing the reduction of the loop by pulling on the rope.

FIGS. 2A and 2B: show their views in longitudinal and crosswise section, respectively, of an example of the device with retention elements aligned adjacent to the flexible component and their retaining structure.

FIG. 2C: shows a perspective of the device as shown in FIGS. 2A and 2B.

FIG. 3A: shows a perspective view of the device, indicating in its interior a toothed sprocket as retainer of the flexible component.

FIGS. 3B, 3C, 3D and 3E: show respective phases of operation of the device, according to the embodiment shown in FIG. 3A.

FIGS. 4A, 4B, 4C and 4D: show respective views in longitudinal section of the release mechanism of the loop formed by the flexible component or rope of the device.

FIGS. 5A and 5B: shows their views of another embodiment of the device, where FIG. 5A is a view of the long hollow component and FIG. 5B is a longitudinal section of this long component with the flexible component or rope partially inserted.

FIGS. 6A and 6B: show, in their sectional and perspective views, respectively, the device of the variant shown in FIGS. 5A and 5B once the flexible component is completely inserted.

FIGS. 7A and 7B: show, in their longitudinal sectional views, the functional phases of the device of the variant shown in FIGS. 5A to 6B.

The FIGS. 8A, 8B, 8C, 8D and 8E: show different views of the device with blocking means near the proximal end of the long hollow component in order to hold the ends of the flexible component. Specifically, FIG. 8A is a longitudinal section of both components with these anchors, FIG. 8B is a perspective of the set of the device, FIGS. 8C and 8C-bis are views in cross section of the anchors in closed and open positions, respectively, and FIGS. 8D and 8E are views in longitudinal section of the anchors also in closed and open positions.

FIG. 8F: shows a view in longitudinal section of the version of the device shown in FIGS. 8A to 8E, showing the use of surgical forceps as the locking system of the flexible component or rope.

FIGS. 9A and 9B: show their perspective views in another embodiment of the device with slots in the hollow component to fasten the ends of the flexible component or rope.

FIGS. 10A, 10B, 10C, 10D, 10E and 10F: shows their perspective views of the device in an embodiment, in which the long hollow component incorporates a dovetail-shaped tray, showing the phases of its use.

FIGS. 11A, 11B and 11C: show views, in longitudinal section, in cross section and in perspective, respectively, of another example of the device, in this case with a clip in the distal end of the long hollow component and in which the flexible component or rope has linear retention elements that slide through a receiving structure located within this clip.

And FIGS. 12A and 12B: show their view in longitudinal section of another example of embodiment of the device, specifically, an example in which the long hollow component incorporates a second hollow component in its interior.

### DETAILED DESCRIPTION OF THE INVENTION

The device to apply compression on the uterine cervix that the invention proposes allows satisfactorily fulfilling the objectives indicated above, with the characterising details that make it possible and that distinguish it appropriately being included in the final claims that accompany this description.

To accomplish this, the device for applying compression on the uterine cervix consists of, essentially, a long hollow component, which has a proximal end and a distal end, and a flexible component in the form of a rope, strap or cord with a first end and a second end, which has a length longer than the length of the long hollow component and whose two ends are inserted into the hollow part of the long hollow component through its distal end, forming a loop with a variable diameter that emerges through this distal end, in order to be wrapped around the object and that applies compression on said object when pulling on at least one of its ends, through the proximal end of the long hollow component.

In one embodiment, the device for applying compression on an object consists of a long hollow component, which has a proximal end and a distal end, and a flexible component or rope, which has a length longer than the length of the long hollow component. The flexible component includes a first end and a second end. The first end of the flexible component is attached to the long hollow component next to the distal end. The second end of the flexible component is inserted in the long hollow component, forming a loop. Through this loop, the flexible component is wrapped around the object and it applies compression force on it when the second end of the flexible component is pulled through the proximal end of the long hollow component.

In another embodiment, the device for applying compression on an object consists of a long hollow component, which has a proximal end and a distal end, and a flexible component or rope, which has a length more than twice the length of the long hollow component. The flexible component includes a first end and a second end. The first end and the second end of the flexible component are configured to be inserted in the long hollow component through the distal end of the long hollow component. The flexible component forms a loop in the distal end and is wrapped around the object and it applies compression force on the object when the first and second ends of the flexible component are pulled at the same time through the proximal end of the long hollow component.

And, in another embodiment, the device for applying compression on an object consists of a long hollow component, which has a proximal end and a distal end, and a clip located at the distal end of the long hollow component, and a flexible component or rope, which has a length longer than the length of the long hollow component. The flexible component includes a first end and a second end; the first end of the flexible component is attached to the clip. The second end of the flexible component is inserted in the long hollow component through the distal end, in which the flexible component is configured for wrapping around the object, and in which the flexible component is configured to compress the object when the second end of the flexible component is pulled.

In any of the three options, the loop is placed around the object, that is, around the cervix immediately before the surgical procedure and, constraining it by pulling on the loop, it functions as a haemostatic tourniquet in order to minimise bleeding, during and after the surgical procedure. The haemostatic tourniquet provides a temporary ischemia to the tissue, decreasing the blood flow from said tissue. The loop also improves the haemostatic process by providing a clean and dry area for the effectiveness of the electro-cauterisation.

### DESCRIPTION OF THE PREFERRED EMBODIMENT

In view of the mentioned Figures, and according to the numeration adopted, one can observe in them various examples of unlimited embodiments of the invented device, which comprises what was indicated and is described in detail below.

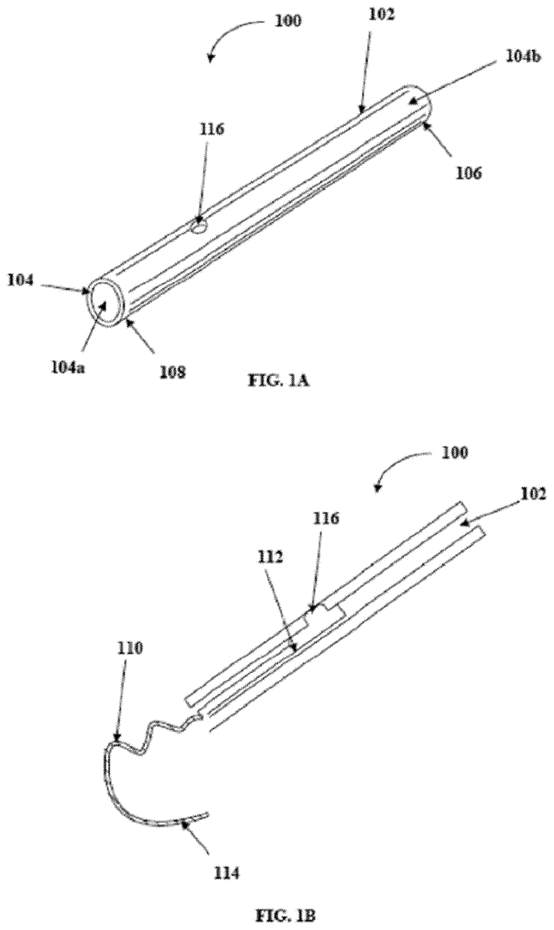

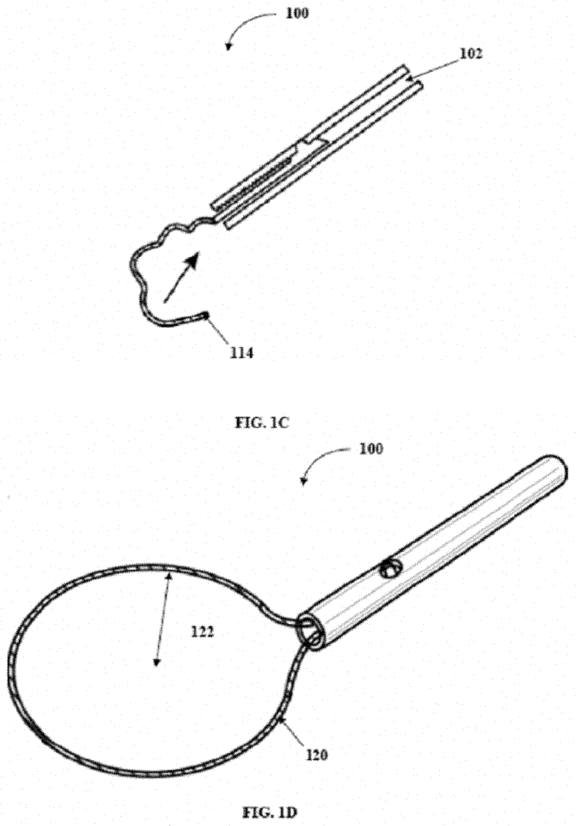

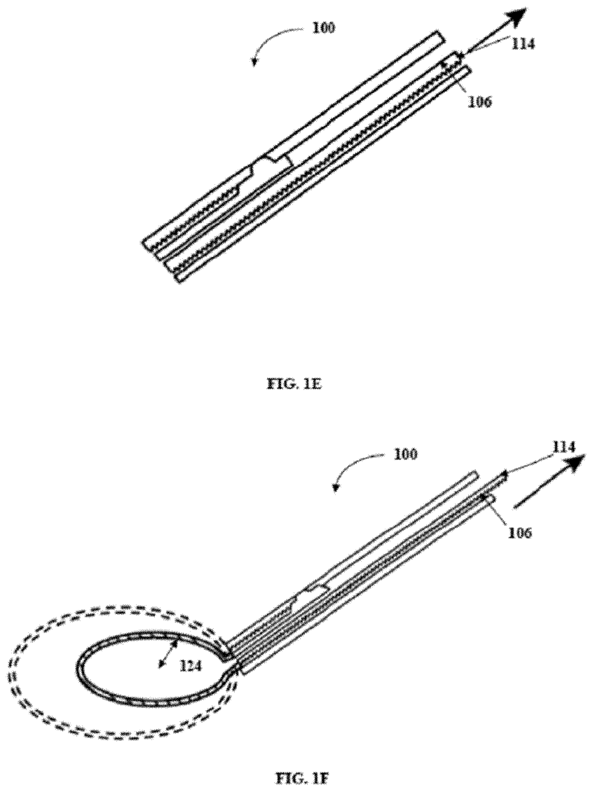

Thus, as is noted in FIGS. 1A, 1B, 1C, 1D, 1E and 1F, they illustrate a preferred embodiment of the device 100 in question, which consists, essentially, a long hollow component 102, which is inserted into the vagina near the cervix, where the compression must be applied, which has a proximal end 106 and a distal end 108, and a flexible component 110 that has a length greater than the length of the long hollow component 102 and which is inserted through its ends 112 and 114 in the hollow of this long hollow component 102 forming a loop 120 of a variable diameter that emerges through the distal end 108 apt for going around the object and applying compression force on it when at least one of the ends 114 of the flexible component 110 is pulled, from the proximal end 106 of the long hollow component 102.

The long hollow component 102, as shown in FIG. 1A, has a first opening 104, an interior wall 104a and an exterior wall 104b. The long hollow component 102 has a proximal end 106 and a distal end 108. And the flexible component 110 has a length greater than the length of the long hollow component 102. The flexible component 110 has a first end 112 and a second end 114. The first opening 104 is extended there through the interior wall 104a of the long hollow component 102 to the exterior wall 104b of the long hollow component 102.

And, in this embodiment, the first end 112 of the flexible component 110 is attached to an anchoring element 116 placed in the exterior wall 104b of the long hollow component 102 with the aid of a tooth-like structure and thereby becoming a fixed part. The second end 114 is free to move forwards or backwards.

As shown in FIGS. 1C and 1D, the second end 114 of the flexible component 110 extends forward and backwards in the interior 104a, creating a mobile or sliding part of the flexible component 110. The second end 114 of the flexible component 110 is inserted towards the back (represented by a black arrow) in the long hollow component 102 through the distal end 108 to form a loop 120 near the distal end 108. The loop 120 has a first diameter 122 as shown in FIG. 1D.

The flexible component 110 is wrapped around the object. As shown in FIGS. 1E and 1F, when the second end 114 of the flexible component 110 is pulled through the proximal end 106 of the long hollow component 102, the first diameter 122, illustrated in FIG. 1D, of the loop 120 decreases to a second diameter 124 and applies compression force on the cervix. The loop 120 is placed around the uterine cervix immediately before the surgical procedure.

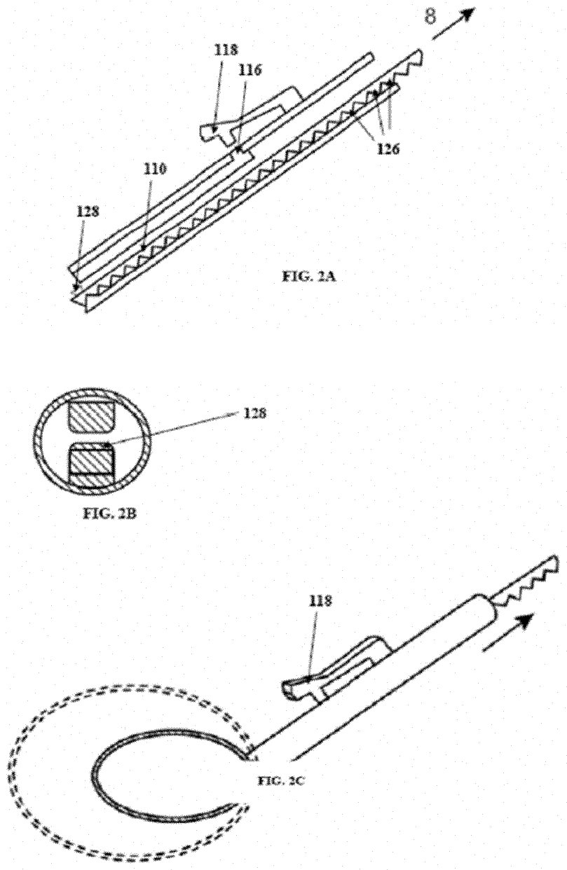

In one embodiment, as shown in FIG. 2A, in a lateral view, the flexible component 110 has a plurality of retention elements 126 aligned adjacent to each other. The plurality of retention elements 126 determines a periodic change in the thickness which forms small teeth or spheres placed along the flexible component 110. In one embodiment, the teeth or spheres are spaced equally throughout the length of the flexible component 110. The retention elements 126 slide through a receiving structure 128 located inside the long hollow component 102. FIG. 2B shows, in a cross-sectional view, the retention elements 126 and the receiving structure 128. And, as is shown in FIG. 2C, the retention elements 126 slide through the receiving structure 128 located within the long hollow component 102 when the second end 114 of the flexible component 110 is pulled through the proximal end 106 of the long hollow component 102. The retention elements 126 ensure that the compression force is applied on the object by the loop 120, and the loop 120 is not undone during the procedure.

In one embodiment, the retention elements 126 are connected to the receiving structure 128, shown in FIG. 2B, impeding the flexible component 110 from sliding outside the distal end 108 when it is pulled. In one embodiment, the retention elements 126 are placed along a half section of the flexible component 110.

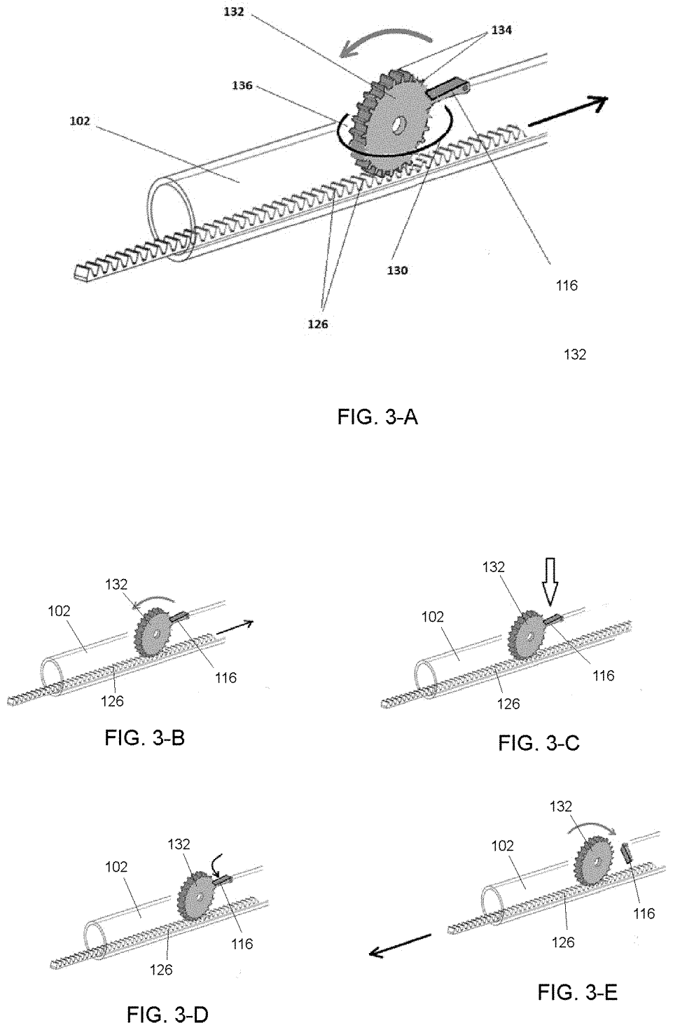

In one embodiment, shown in FIG. 3A to 3E, a housing 130 is placed on the exterior wall 104b of the long hollow component 102. The housing 130 consists of a rotating sprocket gear 132 that has a plurality of teeth 134, where the sprocket 132 is projected in the long hollow component 102 through a second opening 136 that extends through the interior wall 104a of the long hollow component 102 to the exterior wall 104b of the long hollow component 102. The plurality of teeth 134 of the sprocket gear 132 is connected to retention elements 126. The operator can rotate the sprocket using his/her fingers and the connection with the retention elements of the flexible component moves the sliding part thereof, decreasing the diameter of the loop as described in other embodiments.

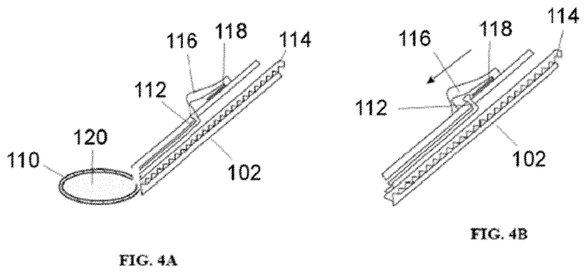

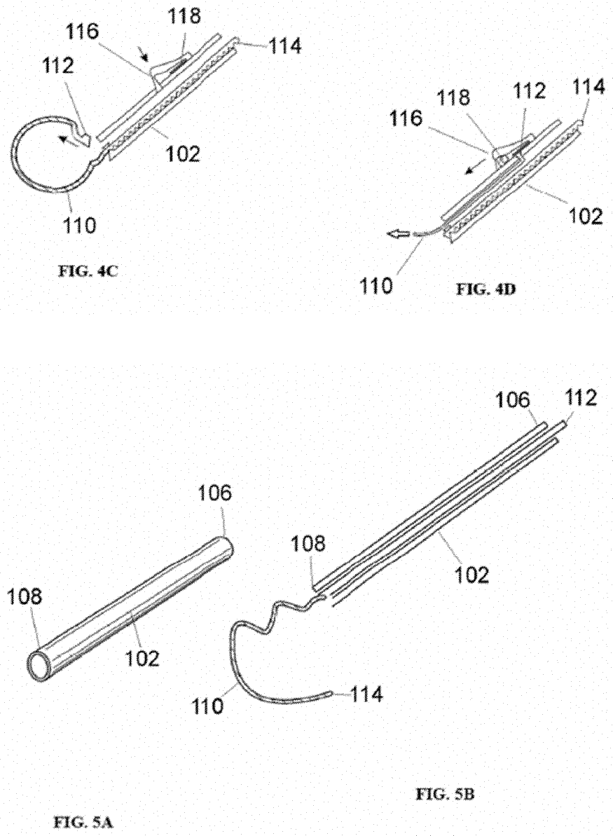

The loop has to be released when compression is not needed; FIGS. 4A, 4B, 4C and 4D illustrate the mechanism of releasing the loop. FIGS. 4A and 4B illustrate the release of the loop 120 for the embodiment shown in FIGS. 1A-1F. FIGS. 4C and 4D illustrate the release of the loop for the embodiment shown in FIGS. 2A-2C, where the flexible component 110 consists of a plurality of retention elements 126 aligned in an adjacent manner. As shown in FIG. 4A, the first end 112 of the flexible component 110 can be joined to an anchoring element 116. In a demonstrable way, the anchoring element 116 can anchor the first end 112 of the flexible component 110 to the first opening 104 in the interior wall 104a of the long hollow component 102.

In some forms of embodiment, as shown in FIG. 4B, the anchoring element 116 has a tooth for pushing the fixed part of the flexible component 110 into the hollow of the long component 102. Force is applied on the anchoring element 116 to push the flexible component outside of the anchoring element 116. Consequently the release mechanism 118 can separate the anchoring element 116 from the first opening 104, loosening in this way the loop 120 as shown in FIG. 4B. In one embodiment, the release mechanism 118 can separate the anchoring element 116 from the first opening 104 cutting a portion of the anchoring element 116 joined to the first opening 104 in the interior wall 104a of the long hollow component 102 as shown in FIG. 4C. In another embodiment, as shown in FIG. 4D, the release mechanism 118 can separate the anchoring element 116 from the first opening 104 by dislodging a part of the anchoring element 116 inserted in the first opening 104 in the interior wall 104a of the long hollow component 102.

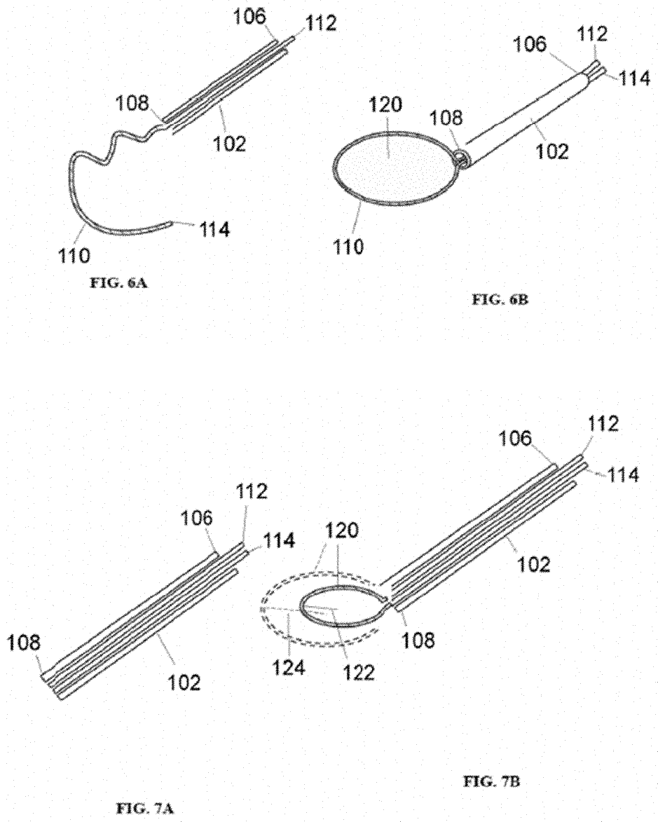

In another embodiment option of the device 100, shown in FIGS. 5A to 7B, the flexible component 110 has a length two times greater than the length of the long hollow component 102. The first end 112 and the second end 114 of the flexible component 110 are inserted in the long hollow component 102 through the distal end 108 of the long hollow component 102 as shown in FIGS. 6A and 6B. The flexible component 110 forms a loop 120 that has a diameter 122 in the distal end 108 as shown in FIG. 6B. The loop 120 is wrapped around the object. As shown in FIG. 7A, the diameter 122 of the loop 120 decreases to 124 when the first end 112 and the second end 114 of the flexible component 110 are pulled together through the proximal end 106 of the long hollow component 102, applying in this way the compression force on the object.

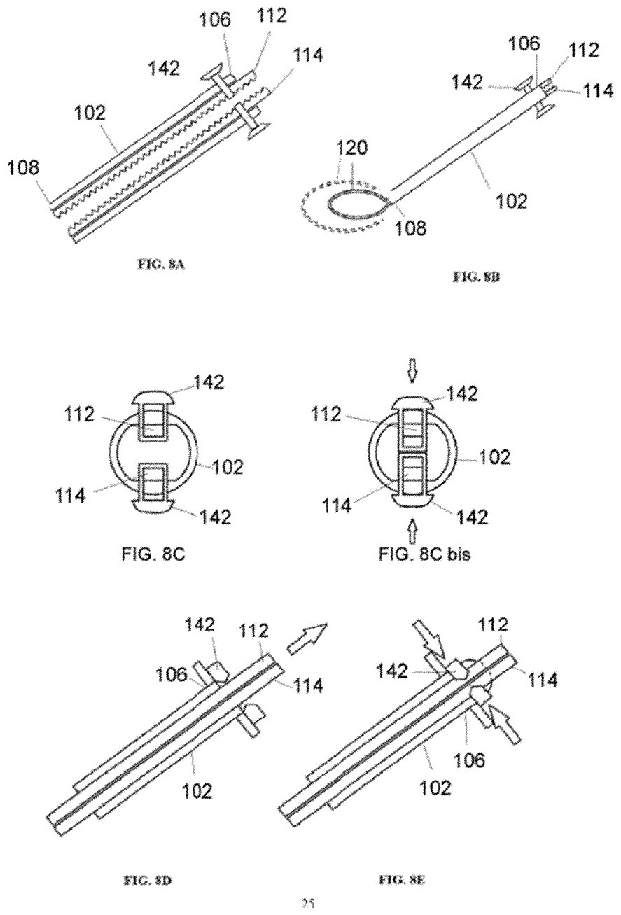

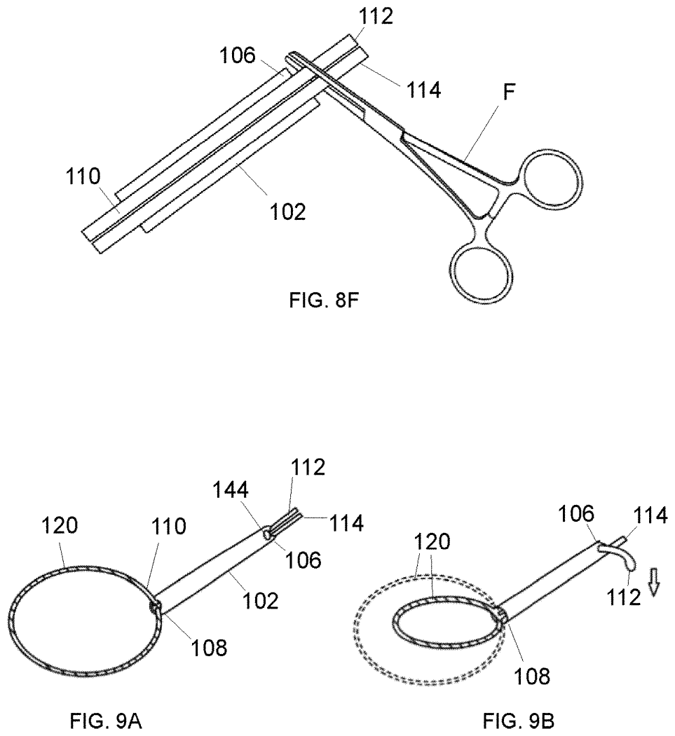

In another embodiment, as shown in FIG. 8A, the first end 112 and the second end 114 of the flexible component 110 are inserted in the long hollow component 102 through the distal end 108 of the long hollow component 102. The first end 112 and the second end 114 slide freely through the long hollow component 102. The long hollow component 102 has a blocking system 142 located on the proximal end 106. This blocking system 142 has a structure similar to the teeth that block the first end 112 and the second end 114 when an external force is applied on it as shown in FIGS. 8C to 8E. In one embodiment, the blocking system is a surgical forceps 142 that can be released when the procedure is finished FIG. 8F.

In another embodiment, as shown in FIGS. 9A and 9B, the first end 112 and the second end 114 of the flexible component 110 are inserted in the long hollow component 102 through the distal end 108 of the long hollow component 102. The first end 112 and the second end 114 slide freely through the long hollow component 102. The long hollow component 102 has one or more slots 144 located in the proximal end 106. One or more slots 144 block one or more of the first end 112 and the second end 114 of the flexible component 110 when the first end 112 and the second end 114 are inserted in one or more slots 144. The flexible component 110 forms the loop 120 which is wrapped around the object and decreases when the first end 112 and the second end 114 of the flexible component 110 are pulled through one or more slots 144 of the long hollow component 102, applying in this way the compression force on the object.

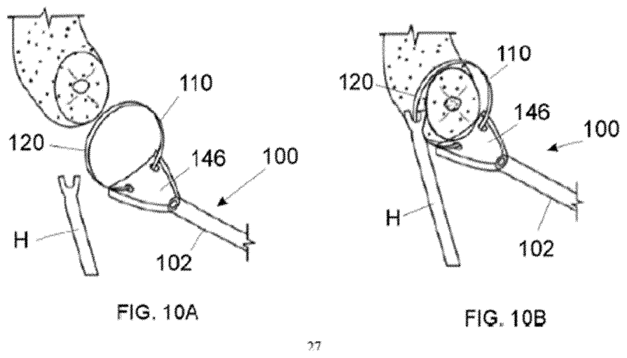

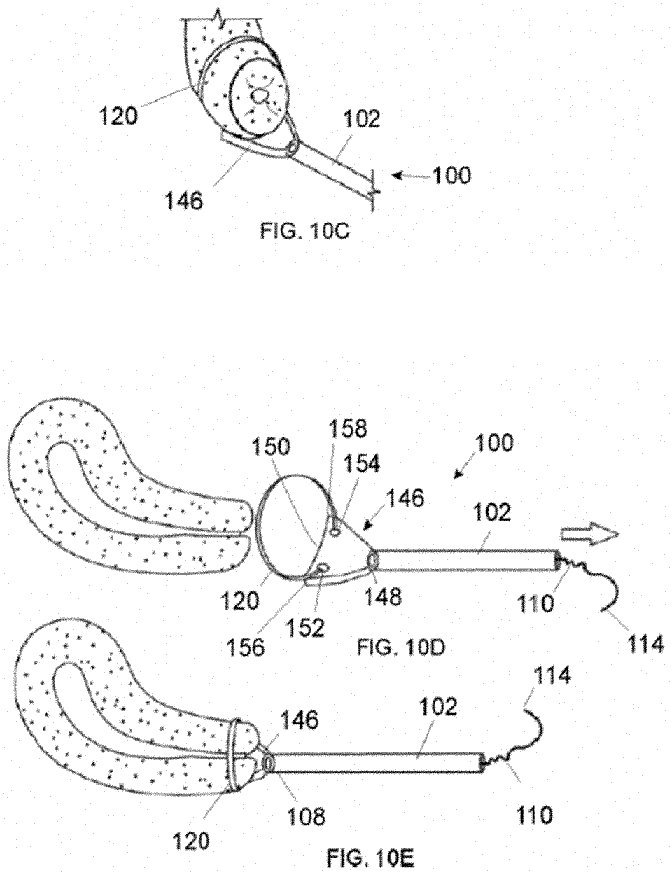

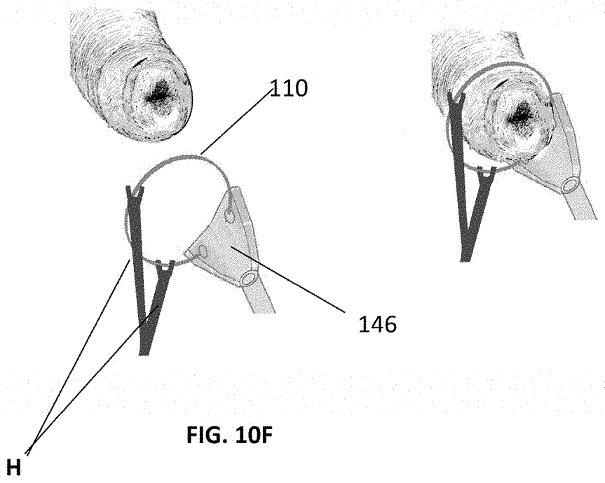

In another embodiment, as shown in FIGS. 10A to 10E, the device 100 has a dovetail-shaped tray 146. A short border 148 of the dovetail-shaped tray 146 is joined to the distal end 108 of the long hollow component 102. The dovetail-shaped tray 146 has a long border 150 situated opposite to the short border 148. The dovetail-shaped tray 146 also has a through-hole 152 and a second through-hole 154 situated in a first end 156 and a second end 158 of the long border 150 of the dovetail-shaped tray 146. The first end 112 and the second end 114 of the flexible component 110 is inserted through the first through-hole 152 and the second through-hole 154 before inserting the first end 112 and the second end 114 of the flexible component 110 in the long hollow component 102 through the distal end 108. The dovetail-shaped tray 146 slides under the object.

As observed in FIGS. 10A, 10B, optionally, to insert the loop 120 of the flexible component 110 around the object. The embodiment has an extra piece (H) consisting in an elongated member with a Y shaped tip in order to help in the placement of the flexible member around the cervix. FIG. 10A, 10B. This extra piece can have two or more Y shaped tips in order to surround the cervix and ease the procedure FIG. 10F.

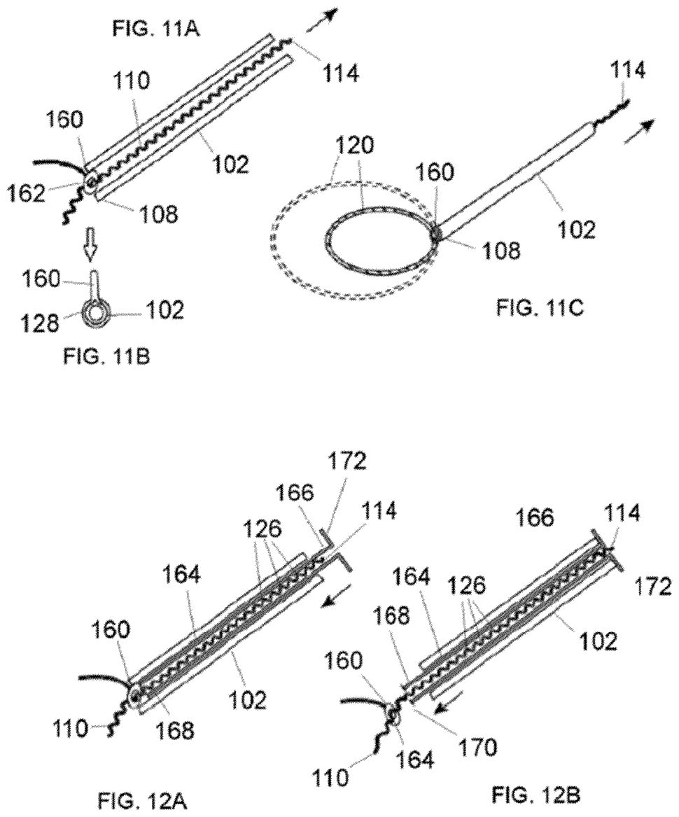

In one embodiment, as shown in FIGS. 11A, 11B and 11C, a clip 160 is situated in the distal end 108 of the long hollow component 102. The first end 112 of the flexible component 110 is joined to the clip 160 and the second end 114 of the flexible component 110 is inserted in the long hollow component 102 through the distal end 108. The clip 160 has a first opening 162. The second end 114 of the flexible component 110 is inserted in the long hollow component 102 through the first opening 162 of the clip 160.

In another embodiment, as shown in FIG. 11B, the clip 160 is situated in the distal end 108 of the long hollow component 102. The first end 112 of the flexible component 110 is joined to the clip 160 and the second end 114 of the flexible component 110 is inserted in the long hollow component 102 through the distal end 108. The flexible component 110 has a plurality of retention elements 126 aligned in an adjacent manner. The retention elements 126 are placed between a middle section and the second end 114 of the flexible component 110. As shown in FIG. 11E, the retention elements 126 slide through a receiving structure 128 situated within the first opening 162 of the clip 160 when the second end 114 of the flexible component 110 is pulled through the proximal end 106 of the long hollow component 102.

The flexible component 110 that has the retention elements 126 slides through the receiving structure 128 when the second end 114 of the flexible component 110 is pulled through the proximal end 106 of the long hollow component 102. The retention elements 126 are connected to the receiver of the structure 128 and impede the flexible component 110 from sliding out of the distal end 108 when the flexible component 110 is withdrawn from the distal end 108. The retention elements 126 have a plurality of teeth or a plurality of spheres. The clip 160 is joined in a way that can be removed at the distal end 108 of the long hollow component 102. The flexible component 110 is wrapped around the object and the compression force is applied on the object when the second end 114 of the flexible component 110 is pulled.

Finally, in another embodiment, as shown in the FIGS. 12A, 12B, the device 100 has a long hollow interior component 164 placed coaxially within the long hollow component 102. The long hollow interior component 164 has a proximal end 166 and a distal end 168. The long hollow interior component 164 has a tab 170 that extends towards the interior in the distal end 168 and a tab 172 that extends outwards at the proximal end 166. As shown in FIG. 12B, the flexible component 110 is inserted in the long hollow interior component 164 through the tab 170 that extends towards the interior and extends through the proximal end 166 that has the tab 172 that extends outwards and detaches the clip 160 when the long hollow interior component 164 slides within the long hollow component 102 through the proximal end 106 of the long hollow component 102.

Having sufficiently described the nature of this invention, as well as the way of putting it into practice, it is not considered necessary to extend its explanation further so that any expert in the subject may understand its scope and the advantages that are derived from it, noting that, within its essentiality, it can be put into practice in other forms of embodiment that differ in detail from that indicated as an example, and to which the protection being sought will also extend as long as its fundamental principle is not altered, changed or modified.

1. Device for applying compression force around the uterine cervix under different conditions in obstetrics and gynaecology, for example, as a tourniquet to stop a haemorrhage, comprising:
a long hollow component (102) that is inserted in the part of the vagina close to the cervix, where the compression must be applied, which has a proximal end (106) and a distal end (108), and
a flexible component (110) that is longer in length than the hollow component (102) and that is inserted by its ends (112, 114) in the interior of the hollow component (102) forming a loop (120) of a variable diameter that emerges through the distal end (108), apt for wrapping around the uterine cervix and applying compression when one or both ends (114) of the rope (110) are pulled from the proximal end (106) of the hollow component (102).

2. The device, according to claim 1, wherein the first end (112) of the flexible component (110) is joined to an anchoring element (116) placed on the exterior face (104b) of the long hollow component (102) with a structure similar to a tooth that converts it into a fixed portion, while the second end (114) remains free to move forwards or backwards.

3. The device, according to claim 2, wherein there is a release mechanism (118) to separate the anchoring element (116).

4. The device, according to claim 3, wherein the flexible component (110) has a plurality of retention elements (126) aligned adjacent to each other which determine a periodic change in the thickness that forms small teeth or spheres placed along the flexible component (110) that are spaced equally along its length.

5. The device, according to claim 4, wherein inside the hollow component (102) there is a receiving structure (128) where the retention elements (126) are connected when they slide through the hollow component (102), preventing the flexible component (110) from sliding out of it.

6. The device, according to claim 4, wherein the exterior face (104b) of the hollow component (102) presents a housing (130) that includes a rotating sprocket gear (132) that has a plurality of teeth (134) that are connected to the retention elements (126).

7. The device, according to claim 6, wherein the sprocket gear (132) is projected in the hollow component (102) through a second opening (136) that extends through the interior face (104a) to the exterior wall (104b) of the long hollow component (102).

8. The device, according to claim 1, wherein the length of the flexible component (110) is two times greater than the length of the hollow component (102) and the first end (112) and the second end (114) of the flexible component (110) slide freely through the hollow component (102) to be pulled together through the proximal end (106) of the hollow component (102) in order to decrease the diameter of the loop (120) which is formed in the distal end (108) and in this way to apply compression force on the cervix.

9. The device, according to claim 8, wherein the hollow component (102) has a blocking system (142) located in the proximal end (106), with a structure similar to teeth, which blocks the first end (112) and the second end (114) of the flexible component (110) when force is applied on it and that can be released using surgical forceps (F).

10. The device, according to claim 1, wherein the hollow component (102) has one or more slots (144) located in the proximal end (106) that block the first end (112) or the second end (114) of the rope (110) when they are inserted in these slots (144).

11. The device, according to claim 1, wherein said device has a dovetail-shaped tray (146) with a short border (148) connected at the distal end (108) of the hollow component (102) in which the flexible component (110) is inserted.

12. The device, according to claim 11, wherein the dovetail-shaped tray (146) has a long border (150) located opposite to the short border (148), and two through-holes (152) and (154) situated at both ends (156) (158) of the long border (150) of the tray in which the first end (112) and the second end (114) of the flexible component (110) are inserted before being inserted again in the hollow component (102) through its distal end (108).

13. The device, according to claim 1, wherein said device has a clip (160) situated at the distal end (108) of the hollow component (102), to which is joined the first end (112) of the flexible component (110) while the second end (114) of the flexible component (110) is inserted in the hollow component (102) through the distal end (108).

14. The device, according to claim 13, wherein the clip (160) has a first opening (162) through which the second end (114) of the flexible component (110) is inserted when it is inserted in the hollow component (102).

15. The device, according to claim 14, wherein the clip (160) is situated at the distal end (108) of the hollow component (102), the first end (112) of the flexible component (110) is connected to the clip (160) and the second end (114) of the flexible component (110) has a plurality of retention elements (126) aligned in an adjacent manner and inserted in the hollow component (102) through the distal end (108) so that the retention elements (126) slide through a receiving structure (128) located within the first opening (162) of the clip (160) when this second end (114) of the flexible component (110) is pulled through the proximal end (106) of the hollow component (102).

16. The device, according to claim 15, wherein there is a long hollow interior component (164), placed coaxially inside the hollow component (102), in which the flexible component (110) is inserted.

17. The device, according to claim 16, characterised in that the long hollow interior component (164) has a proximal end (166) and a distal end (168), a tab (170) that extends towards the interior at the distal end (168) and a tab that extends outwards (172) at the proximal end (166) and the rope (110) is inserted in the long hollow interior component (164) through the tab (170) that is extended towards the interior and is extended through the proximal end (166) that has the tab (172) and removes the clip (160) when the long hollow interior component (164) slides within the hollow component (102) through the proximal end (106) of the hollow component (102).
